(19) United States
(12) Patent Application Publication
(10) Pub. No.: US 2026/0295234 A1
(43) Pub. Date: Oct. 1, 2026

(54) CATHETER BLOOD PUMPS AND ASSOCIATED METHODS

(71) Applicant: Supira Medical, Inc., Los Gatos, CA (US)

(72) Inventor: Daniel HILDEBRAND, Santa Cruz, CA (US)

(21) Appl. No.: 19/479,678

(22) PCT Filed: Apr. 29, 2024
(86) PCT No.: PCT/US2024/026768

§ 371 Date: Oct. 28, 2025

Related U.S. Application Data
(60) Provisional application No. 63/498,923, filed on Apr. 28, 2023.

(51) Int. Cl. A61M 60/174; A61M 60/13; A61M 60/216

(52) U.S. Cl. CPC A61M 60/174; A61M 60/13; A61M 60/216; A61M 2205/3303; A61M 2205/3327; A61M 2205/3334; A61M 2205/3344; A61M 2205/3365; A61M 2205/3368; A61M 2205/502; A61M 2210/125; A61M 2230/207; A61M 2230/30; A61M 2230/50

(57) ABSTRACT

Catheter blood pump positioning techniques, methods, and algorithms are described. In one embodiment, pressure sensor measurements are used to determine the distance to the aortic valve and the left ventricle. An optimal positioning location can be determined based on the pressure sensor measurements. The catheter blood pump can then be optimally positioned to locate the inlet in the ventricle and the outlet in the aorta.

### PRIORITY CLAIM

This patent application claims priority to U.S. Provisional Patent Application No. 63/498,923, titled “CATHETER BLOOD PUMPS AND ASSOCIATED METHODS,” and filed on Apr. 28, 2023, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. Intra-aortic balloon pumps (IABP) are used to support circulatory function, such as treating heart failure patients. An IABP is typically placed within the aorta, and inflated and deflated in counter-pulsation fashion with the heart contractions, with one function being to provide additive support to the circulatory system. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

More recently, minimally invasive rotary blood pumps have been developed, which are inserted into the body in connection with the cardiovascular system to pump arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient. The smallest rotary blood pumps currently available may be percutaneously inserted into the vasculature of a patient through an access sheath, thereby avoiding more extensive surgical intervention, or through a vascular access graft. One such device is a percutaneously inserted ventricular support device.

Although blood pumps exist, there is a need to provide additional improvements in the field of ventricular support devices and similar blood pumps for treating compromised cardiac blood flow.

### SUMMARY OF THE DISCLOSURE

The disclosure is related to intravascular blood pump and methods of their use.

A method is provided, comprising: positioning a catheter blood pump in a heart of a subject; activating an impeller of the catheter blood pump to move blood from an inlet, through a blood conduit, and out of an outlet of the catheter blood pump; continuously or intermittently recording first and second pressure signals with first and second pressure sensors of the catheter blood pump; and estimating a flow rate of the catheter blood pump based on a pressure differential between the first and second pressure signals and one or more polynomial or piecewise polynomial coefficients for a given impeller speed of the catheter blood pump.

In one aspect, the first pressure sensor is positioned near a distal end of the catheter blood pump.

In another aspect, the second pressure sensor is positioned near a proximal end of the catheter blood pump.

In some embodiments, the first pressure sensor is configured to measure a ventricular pressure signal. In another embodiment, the second pressure sensor is configured to measure an aortic pressure signal.

In some aspects, the polynomial coefficients are determined based on real-world testing of the catheter blood pump.

In another aspect, the method comprises accessing the one or more polynomial or piecewise polynomial coefficients from a lookup table.

In another aspect, the method comprises outputting or displaying the estimated flow rate.

In one aspect, the method includes measuring a hematocrit parameter of the subject, wherein estimating the flow rate further comprises estimating the flow rate based on the pressure differential, one or more polynomial or piecewise polynomial coefficients, and the hematocrit parameter.

In another aspect, the method includes measuring a temperature parameter of the subject, wherein estimating the flow rate further comprises estimating the flow rate based on the pressure differential, one or more polynomial or piecewise polynomial coefficients, and the temperature parameter.

A catheter blood pump is provided, comprising an expandable blood conduit defining a lumen between an inlet and an outlet; an impeller at least partially disposed within the blood conduit; a first pressure sensor configured to measure a first pressure signal; a second pressure sensor configured to measure a first pressure signal; one or more processors operatively coupled to the impeller and the first and second pressure sensors, the one or more processors being configured to estimate a flow rate of the catheter blood pump based on a pressure differential between the first and second pressure signals and one or more polynomial or piecewise polynomial coefficients for a given impeller speed of the catheter blood pump.

In some aspects, the first pressure sensor is positioned near a distal end of the catheter blood pump.

In one aspect, the second pressure sensor is positioned near a proximal end of the catheter blood pump.

In other aspects, the first pressure sensor is configured to measure a ventricular pressure signal.

In some aspects, the second pressure sensor is configured to measure an aortic pressure signal.

In additional aspects, the polynomial coefficients are determined based on real-world testing of the catheter blood pump.

In some aspects, the one or more processors are configured to access the one or more polynomial or piecewise polynomial coefficients from a lookup table.

In one aspect, the one or more processors are configured to output or display the estimated flow rate on a display.

In other aspects, the blood pump further includes one or more sensors configured to measure a hematocrit parameter of the subject, wherein the one or more processors are configured to estimate the flow rate based on the pressure differential, one or more polynomial or piecewise polynomial coefficients, and the hematocrit parameter.

In one aspect, the blood pump further includes one or more sensors configured to measure a temperature parameter of the subject, wherein the one or more processors are configured to estimate the flow rate based on the pressure differential, one or more polynomial or piecewise polynomial coefficients, and the temperature parameter.

A non-transitory computing device readable medium having instructions stored thereon, wherein the instructions are executable by a processor to cause a computing device to perform a method comprising: receive an impeller pump speed of a catheter blood pump; receive a first pressure signal from a first pressure sensor of the blood pump; receive a second pressure signal from a second pressure sensor of the blood pump; receive one or more polynomial or piecewise polynomial coefficients based on a given impeller speed; and estimate a flow rate of the catheter blood pump based on a pressure differential between the first and second pressure signals and the one or more polynomial or piecewise polynomial coefficients.

These and other details and aspects are described herein.

### BRIEF DESCRIPTION OF THE DRAWINGS

FIG. 1 is a side view of an exemplary blood pump that includes an expandable scaffold that supports a blood conduit with an impeller housed therein.

FIGS. 2A-2E illustrate one embodiment of a sequence of placing a blood pump in a heart of a patient.

FIGS. 3A-3B are tables showing polynomial coefficients and pump speeds used to estimate flow rate through a catheter blood pump.

FIG. 4 is a chart showing a relationship between flow rate and pressure differential for a plurality of pump speeds.

FIG. 5 is a flowchart describing an algorithm for estimating a catheter blood pump flow rate.

### DETAILED DESCRIPTION

The present disclosure is related to medical devices, systems, and methods of use and manufacture. In particular, described herein are pumps adapted to be disposed within a physiologic vessel, wherein the distal pump portion includes one or more components that act upon fluid. For example, the pumps herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

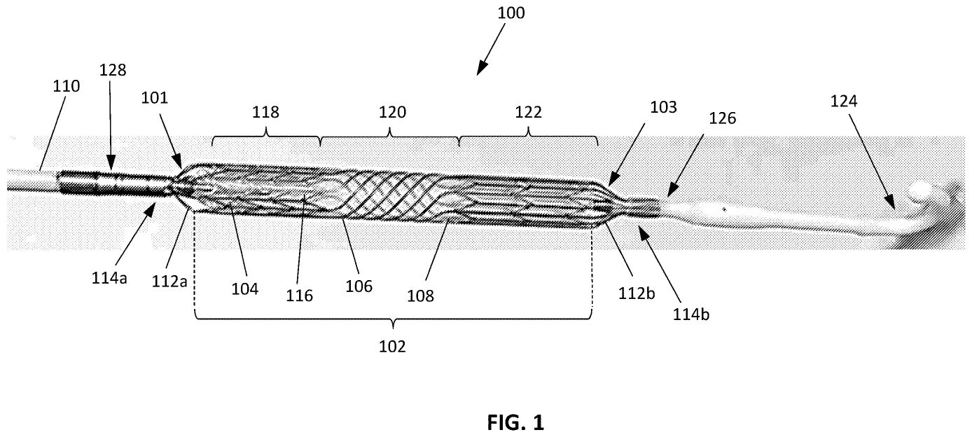

FIG. 1 shows a side view of an exemplary intravascular catheter blood pump 100. The blood pump 100 includes an expandable/collapsible blood conduit 102 that is configured to transition between an expanded state, as shown in FIG. 1, and a collapsed state (not shown). For example, the conduit 102 may be in the collapsed state when confined within a delivery catheter for delivery to the heart, expanded upon release from the delivery catheter for blood pumping, and collapsed back down within the delivery catheter (or other catheter) for removal from heart. When in the expanded state, the conduit 102 is radially expanded so as to form an inner lumen for passing blood therethrough. When in the expanded state, the inner lumen of the conduit 102 may be configured to accommodate blood pumped by one or more impellers therein. The one or more impellers may be collapsible so that they may collapse to a smaller diameter when the conduit 102 is in the collapsed state. The one or more impellers may be positioned within one or more impeller regions of the conduit 102. In some examples, the impeller region(s) of the conduit 102 is/are radially stiffer than other regions (e.g., adjacent regions) of the conduit 102 to prevent the impeller(s) from contacting the interior walls of the conduit 102.

In this example, the blood pump 100 includes an impeller 104 within a proximal portion of the conduit 102. In some cases, the blood pump 100 can include more than one impeller. For example, the blood pump 100 may include a second impeller in a distal region 122 of the fluid conduit 102. In some cases, blood pump 100 may include more than two impellers. The conduit 102 includes a first (e.g., proximal) end having a first (e.g., proximal) opening 101, and a second (e.g., distal) end having a second (e.g., distal) opening 103. The first opening 101 and second opening 103 may be configured as and an inlet and outlet for blood. For example, blood may largely enter the conduit 102 via the second (e.g., distal) opening 103 and exit the conduit 102 via the first (e.g., proximal) opening 101. In such case, the second opening 103 acts as a blood inlet and the first opening 101 acts as a blood outlet. The one or more impellers (e.g., impeller 104) may be configured to pump blood from the inlet toward the outlet. In an exemplary operating position, the second opening 103 (e.g., inlet) may be distal to the aortic valve, in the left ventricle, and the first opening 101 (e.g., outlet) may be proximal to the aortic valve (e.g., in the ascending aorta).

The conduit 102 includes a tubular expandable/collapsible scaffold 106 that provides structural support for a membrane 108 that covers at least a portion of inner surfaces and/or outer surfaces of the scaffold 106. The scaffold 106 includes a material having a pattern of openings with the membrane 108 covering the openings to retain the blood within the lumen of the conduit 102. The scaffold 106 may be unitary and may be made of a single piece of material. For example, the scaffold 106 may be formed by cutting (e.g., laser cutting) a tubular shaped material. Exemplary materials for the scaffold 106 may include one or more of: nitinol, cobalt alloys, and polymers, although other materials may be used.

The blood pump 100 includes proximal struts 112a that extend from the scaffold 106 near the first opening 101 (e.g., blood outlet region) and distal struts 112b that extend from the scaffold 106 near the second opening 103 (e.g., blood inlet region). The proximal struts 112a are coupled to first hub 114a of a proximal shaft 110. The distal struts 112b are coupled to second hub 114b of a distal portion 114. In this example, the first hub 114a includes a bearing assembly through which a central drive cable 116 extends. The drive cable is operationally coupled to and configured to rotate the impeller 104.

In some cases, the impeller 104 is fully positioned axially within the conduit 102. In other cases, a proximal portion of the impeller 104 is positioned at least partially outside of the conduit 102. That is, at least a portion of the impeller may be positioned in axially alignment with a distal portion of the struts 112a.

The conduit 102 and the scaffold 106 may characterized as having a proximal region 118, a central region 120, and a distal region 122. The central region 120 may be configured to be placed across a valve (e.g., aortic valve) such that the proximal region 118 is at least partially within a first heart region (e.g., ascending aorta) and the distal region 122 is at least partially within a second heart region (e.g., left ventricle). The proximal region 118 (and in some cases the distal region 122) may be configured to house an impeller therein. The proximal region 118 may (and in some cases the distal region 122) has a stiffness sufficient to withstand deformation during operation of the blood pump 100 when within the beating heart and to maintain clearance (i.e., a gap) between an impeller region of the blood pump 100 and the rotating impeller 104. The distal region 122 includes the second (e.g., distal) opening 103 of the conduit 102, and may serve as the blood inlet for the conduit 102.

The central region 120 may be less rigid relative to the proximal region 118 (and in some cases the distal region 122). The higher flexibility of the central region 120 may allow the central region 120 to deflect when a lateral force is applied on a side of the conduit 102, for example, as the conduit 102 traverses through the patient's blood vessels and/or within the heart. For example, the central region 120 may be configured to laterally bend upon a lateral force applied to the distal region 122 and/or the proximal region 118. In some cases, it may be desirable for the central region 120 to laterally bend as the conduit 102 traverses the ascending aorta and temporarily assume a bent configuration when the conduit 102 is positioned across an aortic valve. In this example, the central region 120 includes a helical arrangement of longitudinally running elongate elements configured to provide flexibility for lateral bending. In some examples, a distal tip 124 of the blood pump 100 is curved to form an atraumatic tip. In some cases, the distal tip 124 flexible (e.g., laterally bendable) to enhance the atraumatic aspects of the distal tip 124. For example, the distal tip 124 may be sufficiently flexible to bend when pressed against tissue (e.g., by a predetermined amount of force) to prevent puncture of the tissue.

The first hub 114a (e.g., proximal hub) and/or the second hub 114b (e.g., distal hub) may include features that promote smooth blood flow into and/or out of the conduit 102. Such features may prevent or reduce the occurrence of stagnant and/or turbulent blood flow that may otherwise tend to occur in regions near the first opening 101 (e.g., outlet region) and/or the second opening 103 (e.g., inlet region) of the conduit 102. Since stagnant and/or turbulent blood flow is associated with blood coagulation and/or clotting, measures to reduce this can be beneficial to patient outcomes.

The blood pump 100 can include one or more pressure sensors configured to take a pressure reading inside the patient. In some embodiments, the blood pump 100 can include a distal pressure sensor 126 positioned near or adjacent to the inlet or distal hub 114b, and a proximal pressure sensor 128 positioned near or adjacent to the outlet or proximal hub 114a. The pressure sensors can be any pressure sensor known in the art. In some embodiments, the pressure sensors can comprise MEMs pressure sensors. In some embodiments, the pressure sensors can comprise fluid column or fiber optic sensors. In some embodiments, at least one sensor is a flow sensor for directly measuring volumetric flow rate. In some embodiments, the distal (inlet) and proximal (outlet) pressure sensors are different.

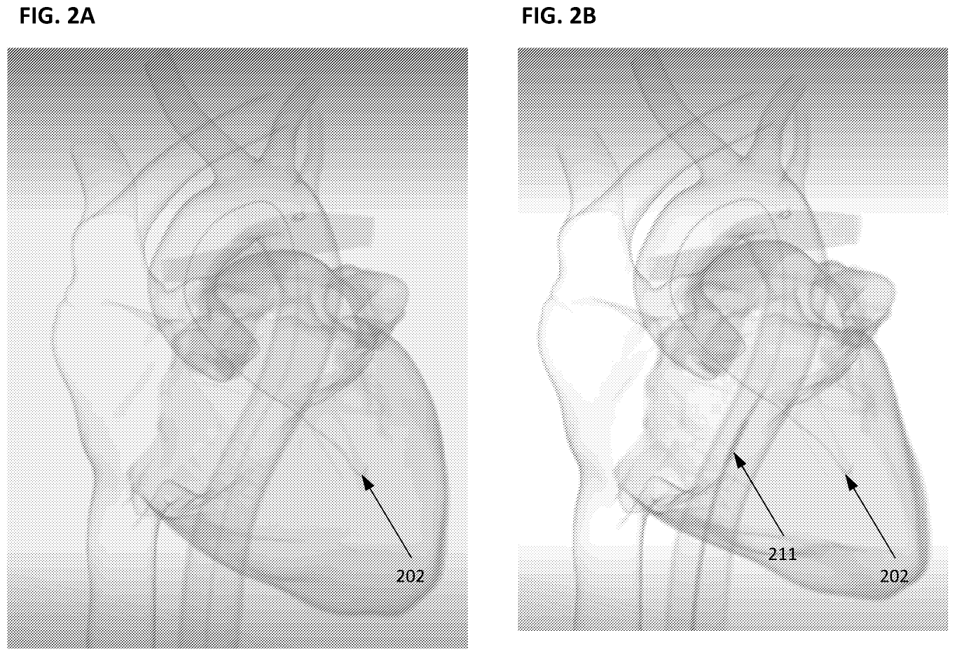

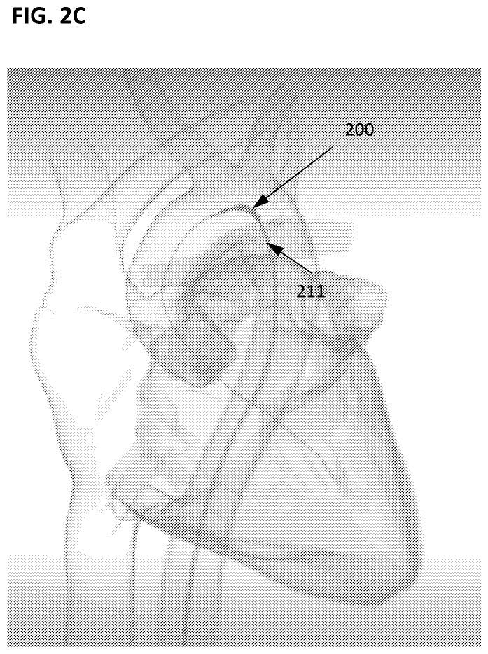

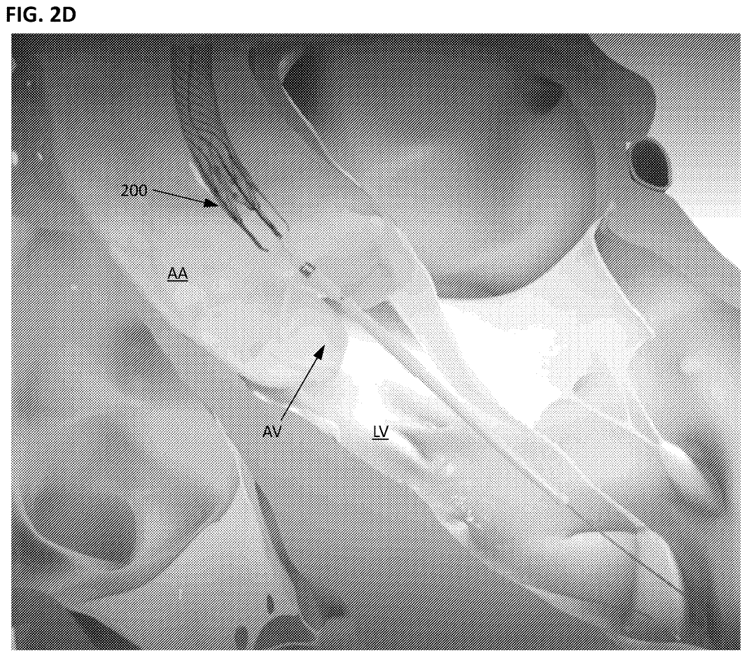

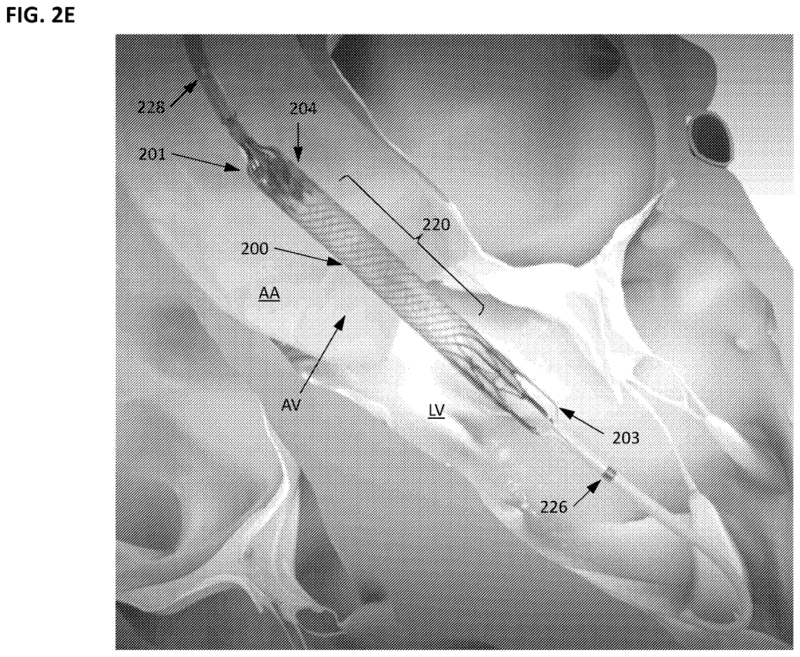

Methods of delivering and placing the blood pump will now be discussed. First, access to the patient's vasculature can be obtained (e.g., without limitation, via femoral access) using an appropriately sized introducer sheath. Using standard valve crossing techniques, in FIG. 2A a guide wire 202 may be advanced until it is positioned securely in the target location (e.g., left ventricle). The guide wire positioning can be performed under imaging such as fluoroscopy

Next, referring to FIG. 2B, the blood pump, positioned in a compressed or delivery configuration within an introducer sheath 211, can be advanced over the guide wire towards a target location.

In FIG. 2C, the introducer sheath 211 can be retracted, exposing the pump portion and causing the pump to expand from the compressed or delivery configuration to an expanded configuration. In some examples, the pump can be expanded in the aorta near the bifurcation to the iliac arteries. The introducer sheath can be long enough to extend to the aorta to allow for expansion of the pump in the aorta. Conventional introducer sheaths are not long enough to enable unsheathing of a pump in this location.

In FIG. 2D, the expanded blood pump 200 can be advanced over the guidewire 202, first into the ascending aorta (“AA”), and then in FIG. 2E, to the target location in which the blood pump extends across the aortic valve (“AV”) and into the left ventricle (“LV”). In FIG. 2E, the blood pump inlet 203 is positioned in the LV and the blood pump outlet 201 and proximal impeller 204 are positioned in the ascending aorta AA. The central region 220 of the blood pump, which may be more flexible than the impeller regions, as described in more detail above, spans the aortic valve AV. With the catheter blood pump in this position, the distal pressure sensor 226 and proximal pressure sensor 228 previously described can be used to measure ventricular and aortic pressures, respectively.

When the pump is in place within the heart, operation of the impeller 204 causes blood to flow from the LV into inlet 203, along the blood conduit of the blood pump, and out of the outlet 201 into the AA to provide cardiac support for the patient. While the blood pump is implanted and in operation, numerous metrics can be monitored to assess patient health, cardiac function, and efficacy of the blood pump, including but not limited to the flow rate of blood through the catheter blood pump. However, dedicated flow sensors tend not to be accurate in cardiac blood pump applications and/or can be difficult to place within the pump blood conduit, or attach to the blood pump itself due to the small size, need for compressibility in expandable pumps, and limited space within the pump to place the sensor and route the electrical wires without adversely affecting operation of the blood pump.

The present disclosure provides systems and methods for calculating an estimated flow rate of the catheter blood pump without requiring a dedicated flow rate sensor. In some embodiments, the flow rate of the catheter blood pump can be determined, calculated, or estimated by one or more processors operatively coupled to the catheter blood pump. For example, the one or more processors may be disposed within a console of the catheter blood pump.

In one implementation, a flow rate estimate of the catheter blood pump, Qₑₛₜ, in liters per minute, can be calculated as a polynomial or piecewise polynomial function of x, the delta pressure P_{delta} in mmHg which is the pressure difference between a distal pressure sensor reading (P_{ventricle}) and a proximal pressure sensor reading (Pₐₒᵣₜₐ), and polynomial coefficients, cₙ, which are a function of the pump's RPM. In one implementation, the polynomial coefficients are determined with real-world testing and measurement of flow rate through the catheter blood pump at a variety of pump speeds (e.g., measured in a lab, in animal studies, or in human studies) at various stages in the manufacturing process (e.g., before and after reliability and simulated-use testing). The polynomial or piecewise polynomial coefficients in this example can be unique for each individual catheter blood pump that is manufactured. In another embodiment, the polynomial or piecewise polynomial coefficients are not unique to individual catheter blood pumps, but instead are determined for a specific catheter blood pump design. In yet another embodiment, the polynomial coefficients can be a function of the blood pump's operating conditions (e.g., blood viscosity, hemocrit, temperature, etc.). For example, some embodiments can assume a fixed blood viscosity. It may be possible to improve the accuracy of the flow rate estimation by measuring some of the actual fluid properties (e.g., viscosity) which is strongly correlated to hematocrit and temperature of the blood. Measuring the hematocrit and/or temperature may optionally enable the system/method to better estimate the actual viscosity, resulting in extra dimensions to the H-Q curves; i.e. different polynomial coefficients for a given speed for a range of blood viscosities.

In one example, a first polynomial coefficient co can correspond to an offset and a second polynomial coefficient ci can correspond to a slope.

Equation 1 below is a 3^{rd} order polynomial function that provides an estimated flow rate Qₑₛₜ for a plurality of pump operating speeds of a catheter blood pump:

$${Q}_{est} - {c}_{3} {x}^{3} + {c}_{2} {x}^{2} + {c}_{1} {x}^{1} + {c}_{0} {x}^{0} \qquad ( Equation 1 )$$

Equation 2 defines x as:

$$x = {P}_{delta} = P {a}_{ortic} - {P}_{ventricle} \qquad ( Equation 2 )$$

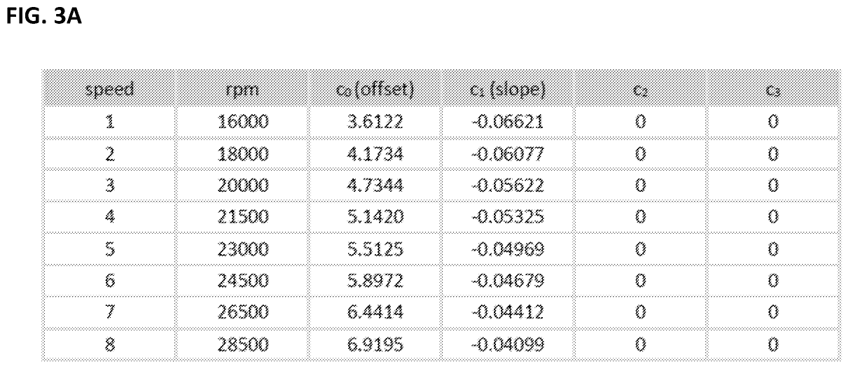

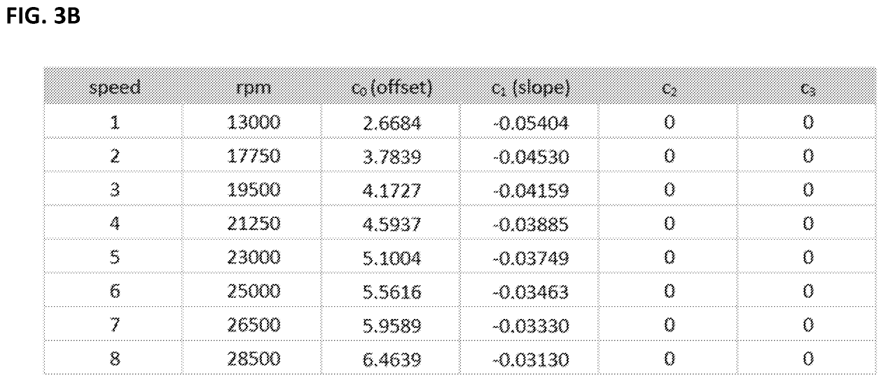

FIG. 3A is a first table showing a plurality of pump speeds of a catheter blood pump, the impeller RPM coinciding with each pump speed, and the polynomial coefficients (determined above) to be used with equation 1. In this example, the catheter blood pump is designed and configured to operate at 8 or more discrete pump speeds. In one specific embodiment, the pump speeds can range from 16000 RPM to 28500 RPM or more. The pump speed is typically chosen by the surgeon/physician specific to the requirements and level of support that the individual patient requires. In the example of FIG. 3A, the pump speeds and corresponding rpms can include: pump speed 1=16000 rpm; pump speed 2=18000 rpm; pump speed 3=20000 rpm; pump speed 4=21500 rpm; pump speed 5=23000 rpm; pump speed 6=24500 rpm; pump speed 7=26500 rpm; and pump speed 8=28500 rpm.

FIG. 3B is a second table showing a plurality of pump speeds of a catheter blood pump, the impeller RPM coinciding with each pump speed, and the polynomial coefficients (determined above) to be used with equation 1. The polynomial coefficients shown in FIG. 3B are different than those of FIG. 3A to reflect specific design changes between pump designs. For example, factors including length, flexibility, diameter, shroud design, impeller design, pump speeds, etc. can all affect the values of the polynomial coefficients required to produce accurate flow estimates. In this example, the catheter blood pump is designed and configured to operate at 8 or more discrete pump speeds. In one specific embodiment, the pump speeds can range from 16000 RPM to 28500 RPM or more. The pump speed is typically chosen by the surgeon/physician specific to the requirements and level of support that the individual patient requires. In the example of FIG. 3A, the pump speeds and corresponding rpms can include: pump speed 1=13000 rpm; pump speed 2=17750 rpm; pump speed 3=19500 rpm; pump speed 4=21250 rpm; pump speed 5=23000 rpm; pump speed 6=25000 rpm; pump speed 7=26500 rpm; and pump speed 8=28500 rpm.

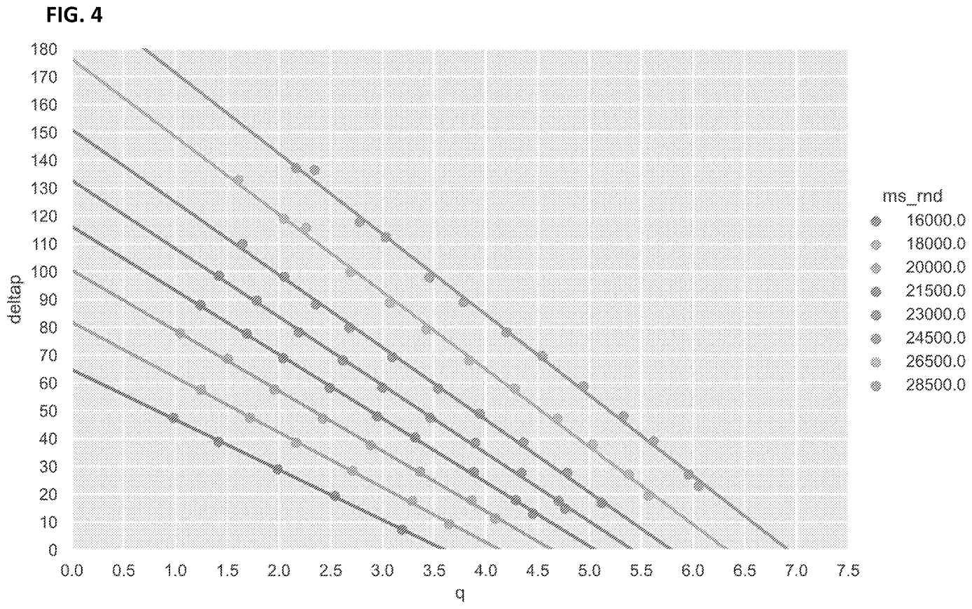

FIG. 4 is a chart showing a linear response between flow rate Q and P_{delta} for each of a plurality of pump speeds. To produce the coefficients of FIG. 3A, the data can be individually fit using a least-squares regression to a 1^{st} order polynomial at each motor speed to produce multiple sets of coefficients for each motor speed. These coefficients can then be averaged to produce the values in the table of FIG. 3A.

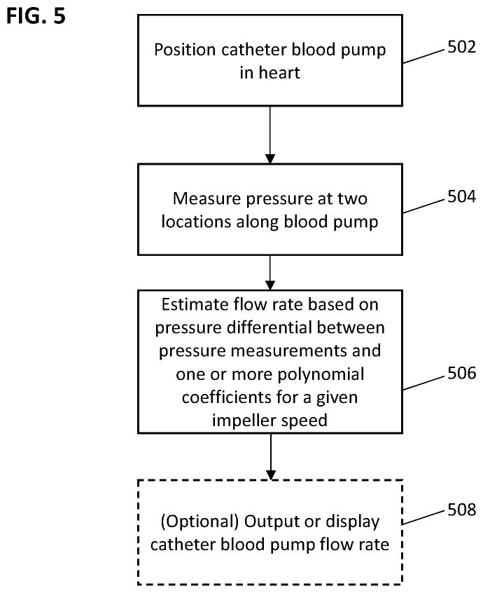

FIG. 5 is a flowchart describing a method or algorithm for estimating a flow rate through a catheter blood pump disposed in the heart. In some embodiments, the method can be implemented in one or more processors, such as in a console connected to the blood pump, configured to execute instructions such as software or firmware to run an algorithm that estimates the flow rate through the blood pump.

At step 502, the catheter blood pump can be positioned in the heart. This can include for example, positioning according to the description above related to FIGS. 2A-2E in which an inlet and distal pressure sensor of the catheter blood pump are disposed in the ventricle and an outlet and proximal pressure sensor are disposed in the aorta.

At step 504, the method can include measuring pressure at two locations along the blood pump. As described above, one embodiment of the blood pump can have a distal pressure sensor at or near the inlet and a proximal pressure sensor at or near the outlet. In some examples, this can result in a first pressure sensor being configured to measure a ventricular pressure signal and a second pressure sensor being configured to measure an aortic pressure signal.

At step 506, the method can further include estimating a flow rate through the blood pump (such as a blood flow rate) based on a pressure differential between the two pressure measurements and one or more polynomial or piecewise polynomial coefficients for a given impeller speed, and optionally, the patient's measured hematocrit and/or blood temperature to estimate the blood viscosity. As described above, in some embodiments the polynomial or piecewise polynomial coefficients can be measured or determined based on real-world testing of each individual catheter blood pump. In other embodiments, the polynomial or piecewise polynomial coefficients can be standardized for a specific blood pump model. The polynomial or piecewise polynomial coefficients can be, for example, stored in a lookup table either on the catheter blood pump itself, in the blood pump console, or alternatively on the cloud. In some embodiments, the method/algorithm can include estimating the flow rate using the equations described above. In some examples, the polynomial coefficients for a given pump speed are shown in the tables of FIGS. 3A-3B.

At step 508, the method can optionally include the step of outputting or displaying the estimated catheter blood pump flow rate. For example, the flow rate can be displayed either continuously or intermittently to a user, such as on a display of the console of the blood pump. Alternatively, the flow rate can be displayed on an external device, such as on a smartphone, pc, or tablet. In some embodiments, the flow rate is output to a remote server, optionally along with other measurements or patient metrics. For example, the processing (at a remote server, clinical console, or otherwise) may make use of various other parameters like hematocrit, temperature, and the like.

Any of the blood pumps described herein may include surfaces with one or more anticoagulant agents. For example, at least a portion of one or more of the hubs, conduits (e.g., scaffold and/or membrane), struts (e.g., proximal and/or distal struts), distal tips and/or impellers of the blood pumps described herein may include a coating or material having an anticoagulant agent. In some cases, the anticoagulant agents may include drugs such as heparin, warfarin and/or prostaglandins.

What is claimed is:

1. A method, comprising:
positioning a catheter blood pump in a heart of a subject;
activating an impeller of the catheter blood pump to move blood from an inlet, through a blood conduit, and out of an outlet of the catheter blood pump;
continuously or intermittently recording first and second pressure signals with first and second pressure sensors of the catheter blood pump; and
estimating a flow rate of the catheter blood pump based on a pressure differential between the first and second pressure signals and one or more polynomial or piecewise polynomial coefficients for a given impeller speed of the catheter blood pump.

2. The method of claim 1, wherein the first pressure sensor is positioned near a distal end of the catheter blood pump.

3. The method of claim 2, wherein the second pressure sensor is positioned near a proximal end of the catheter blood pump.

4. The method of claim 1, wherein the first pressure sensor is configured to measure a ventricular pressure signal.

5. The method of claim 4, wherein the second pressure sensor is configured to measure an aortic pressure signal.

6. The method of claim 1, wherein the polynomial coefficients are determined based on real-world testing of the catheter blood pump.

7. The method of claim 1, further comprising accessing the one or more polynomial or piecewise polynomial coefficients from a lookup table.

8. The method of claim 1, further comprising outputting or displaying the estimated flow rate.

9. The method of claim 1, further comprising measuring a hematocrit parameter of the subject, wherein estimating the flow rate further comprises estimating the flow rate based on the pressure differential, one or more polynomial or piecewise polynomial coefficients, and the hematocrit parameter.

10. The method of claim 1, further comprising measuring a temperature parameter of the subject, wherein estimating the flow rate further comprises estimating the flow rate based on the pressure differential, one or more polynomial or piecewise polynomial coefficients, and the temperature parameter.

11. A catheter blood pump, comprising:
an expandable blood conduit defining a lumen between an inlet and an outlet;
an impeller at least partially disposed within the blood conduit;
a first pressure sensor configured to measure a first pressure signal;
a second pressure sensor configured to measure a first pressure signal;
one or more processors operatively coupled to the impeller and the first and second pressure sensors, the one or more processors being configured to estimate a flow rate of the catheter blood pump based on a pressure differential between the first and second pressure signals and one or more polynomial or piecewise polynomial coefficients for a given impeller speed of the catheter blood pump.

12. The blood pump of claim 11, wherein the first pressure sensor is positioned near a distal end of the catheter blood pump.

13. The blood pump of claim 12, wherein the second pressure sensor is positioned near a proximal end of the catheter blood pump.

14. The blood pump of claim 11, wherein the first pressure sensor is configured to measure a ventricular pressure signal.

15. The blood pump of claim 14, wherein the second pressure sensor is configured to measure an aortic pressure signal.

16. The blood pump of claim 11, wherein the polynomial coefficients are determined based on real-world testing of the catheter blood pump.

17. The blood pump of claim 11, wherein the one or more processors are configured to access the one or more polynomial or piecewise polynomial coefficients from a lookup table.

18. The blood pump of claim 11, wherein the one or more processors are configured to output or display the estimated flow rate on a display.

19. The blood pump of claim 11, wherein the blood pump further includes one or more sensors configured to measure a hematocrit parameter of the subject, wherein the one or more processors are configured to estimate the flow rate based on the pressure differential, one or more polynomial or piecewise polynomial coefficients, and the hematocrit parameter.

20. The blood pump of claim 11, wherein the blood pump further includes one or more sensors configured to measure a temperature parameter of the subject, wherein the one or more processors are configured to estimate the flow rate based on the pressure differential, one or more polynomial or piecewise polynomial coefficients, and the temperature parameter.

21. A non-transitory computing device readable medium having instructions stored thereon, wherein the instructions are executable by a processor to cause a computing device to perform a method comprising:
receive an impeller pump speed of a catheter blood pump;
receive a first pressure signal from a first pressure sensor of the blood pump;
receive a second pressure signal from a second pressure sensor of the blood pump;
receive one or more polynomial or piecewise polynomial coefficients based on a given impeller speed; and
estimate a flow rate of the catheter blood pump based on a pressure differential between the first and second pressure signals and the one or more polynomial or piecewise polynomial coefficients.
